(19) United States
(12) Patent Application Publication
(10) Pub. No.: US 2023/0239635 A1
(43) Pub. Date: Jul. 27, 2023

(54) METHOD FOR ADAPTING A PLURALITY OF SIGNAL PROCESSING PARAMETERS OF A HEARING INSTRUMENT IN A HEARING SYSTEM

(71) Applicant: Sivantos Pte. Ltd., Singapore (SG)

(72) Inventors: Cornelia Wiedenbrüg, Erlangen (DE); Ronny Hannemann, Erlangen (DE); Nadja Schinkel-Bielefeld, Erlangen (DE); Marko Lugger, Erlangen (DE)

(21) Appl. No.: 18/158,097

(22) Filed: Jan. 23, 2023

(30) Foreign Application Priority Data
Jan. 25, 2022 (DE) 10 2022 200 810.3

(51) Int. Cl. H04R 25/00

(52) U.S. Cl. CPC H04R 25/505; H04R 25/558; H04R 25/604; H04R 25/609; H04R 2225/41; H04R 2460/07; H04R 2225/55; H04R 2225/39; A61B 5/02416

(57) ABSTRACT

A method for adapting signal processing parameters of a hearing instrument of a hearing system. A number of acoustic indicators (IndA) for a temporary environmental situation of a user of the hearing instrument is ascertained from an input signal generated by a transducer. A first sensor ascertains a number of peripheral indicators (IndP) for the existing temporary environmental situation. Based on the acoustic and peripheral indicators (IndA, IndP) the system determines whether the existing temporary environmental situation falls into a known class of temporary environmental situations. If it does not, a new class is defined with the respective ascertained acoustic and peripheral indicator(s) (IndA, IndP), and the plurality of signal processing parameters are assigned a corresponding plurality of parameter values, according to which the first input signal is to be processed to form the output signal upon the future existence of a temporary environmental situation from the new class.

### CROSS-REFERENCE TO RELATED APPLICATION

This application claims the priority, under 35 U.S.C. § 119, of German Patent Application DE 10 2022 200 810.3, filed Jan. 25, 2022; the prior application is herewith incorporated by reference in its entirety.

### FIELD AND BACKGROUND OF THE INVENTION

The invention relates to a method in a hearing system for adapting a plurality of signal processing parameters of a hearing instrument of the hearing system. At least one first input signal, which is generated by a first input transducer of the hearing instrument from an ambient sound, is processed to form an output signal, which is converted by an output transducer of the hearing instrument into an output sound signal. On the basis of the first input signal and/or on the basis of a further input signal, which is generated by a further input transducer of the hearing system from the ambient sound, a number of acoustic indicators for an existing environmental situation of a user of the hearing instrument is ascertained. A new class is defined for an environmental situation and in this case the plurality of signal processing parameters are each assigned a corresponding plurality of parameter values, according to which the first input signal is to be processed upon a later presence of an environmental situation from the new class to form an output signal.

In a hearing aid, which is provided for the treatment of a hearing-impaired person, one or more input signals are usually generated by one or more transducers (for example one or more microphones) from an ambient sound. Each input signal is subsequently processed, in particular in a user-specific manner on the basis of the audiological specifications of a user of the hearing aid, wherein individual signal components of the input signal(s) can be amplified and/or compressed, for example, depending on the frequency band. By means of this signal processing, an output signal is generated from the input or signal(s) and this is converted by an output transducer (for example a loudspeaker) into an output sound.

It is often typical in this case that an individual adaptation takes place for the user of the hearing aid in regard to which parameter values of individual signal processing parameters (e.g., amplification factors, compression ratios or knee points of said frequency band-dependent amplification or compression, but also parameters for suppressing interference noises or acoustic feedback) are assigned in dependence on the input signal(s). Individual parameter values of specific signal processing parameters can also be assigned in particular in dependence on so-called hearing situations. Individual classes of typical acoustic environments are defined by these hearing situations, so that if such a hearing situation currently exists, a priori the same predefined parameter values (as a so-called hearing program) are always applied for the signal processing parameters.

A tuning in regard to how individual parameter values are to be applied in specific hearing situations is usually to be carried out by a hearing aid acoustician or the like. This is linked to an effort for the user (since the user has to visit the hearing aid acoustician).

Increasingly, hearing instruments which are not primarily provided for the treatment of hearing-impaired persons, for example, earbuds or wireless ear headphones, are also nonetheless used to be able to offer assistance to the user in hearing up to a certain degree. However, these are not primarily designed in particular for the above-mentioned use in dependence on hearing situations and a trip to the hearing aid acoustician would also probably be required here if so.

### SUMMARY OF THE INVENTION

It is accordingly an object of the invention to provide a method which overcomes the above-mentioned and other disadvantages of the heretofore-known devices and methods of this general type and which provides for a method by means of which signal processing parameters of a hearing instrument may be adapted as easily as possible and by the user himself, so that the signal processing parameters are matched individually to the user by the adaptation for an environment-dependent operation.

With the above and other objects in view there is provided, in accordance with the invention, a method in a hearing system for adapting a plurality of signal processing parameters of a hearing instrument of the hearing system, the method comprising:

processing a first input signal, which is generated by a first input transducer of the hearing instrument from an ambient sound, to form an output signal, and converting the output signal by an output transducer of the hearing instrument into an output sound signal;

determining, from the first input signal, a number of acoustic indicators for an existing temporary environmental situation of a user of the hearing instrument;

determining, based on at least one first sensor of the hearing system, a number of peripheral indicators for the existing temporary environmental situation;

on a basis of one or more of the acoustic indicators and on a basis of one or more of the peripheral indicators, ascertaining whether the existing temporary environmental situation falls in a class from a plurality of known classes for temporary environmental situations; and

when the existing temporary environmental situation does not fall into any of the known classes for temporary environmental situations:
defining a new class for temporary environmental situations, the new class being characterized by the respectively ascertained acoustic indicators and peripheral indicators; and
assigning to the plurality of signal processing parameters a corresponding plurality of parameter values, according to which the first input signal is to be processed to form the output signal upon a later existence of a temporary environmental situation from the new class.

In other words, the objects of the invention are achieved by a method for a hearing system for adapting a plurality of signal processing parameters of a hearing instrument of the hearing system, according to which at least one first input signal, which is generated by a first, in particular electroacoustic input transducer of the hearing instrument from an ambient sound, is processed to form an output signal, which is converted by an in particular electroacoustic output transducer of the hearing instrument into an output sound signal.

It is provided in this case that by means of the first input signal, a number of acoustic indicators is/are ascertained for an existing temporary environmental situation of a user of the hearing instrument, by means of at least one first sensor of the hearing system a number of peripheral indicators for the existing temporary environmental situation is ascertained, and the acoustic indicator(s) and the peripheral indicator(s) are used to ascertained whether the existing temporary environmental situation falls in a class of a plurality of known classes for temporary environmental situations.

Furthermore, it is provided that in the case that the existing temporary environmental situation does not fall in any of the known classes for temporary environmental situations, a new class for a temporary environmental situation is defined which is characterized by the respective ascertained acoustic and peripheral indicator(s), and in this case the plurality of signal processing parameters is each assigned a corresponding plurality of parameter values, according to which the first input signal is to be processed to form an output signal if a temporary environmental situation from the new class exists later. Advantageous embodiments, which are partially inventive considered as such, are the subject matter of the dependent claims and the following description.

In other words, the invention provides a possibility for adapting a plurality of signal processing parameters of a hearing instrument, which are used in running operation of the hearing instrument to process at least one first input signal of the hearing system generated in the described manner to form an output signal to be reproduced in the described manner. The method is carried out here by means of a hearing system. The hearing system at least also comprises the hearing instrument here, and can possibly comprise still further devices, in particular an auxiliary device that is connectable to the hearing instrument.

In this case, initially acoustic indicators and peripheral, thus in particular non-acoustic indicators are ascertained, which supply information about an existing temporary environmental situation of the user. Such a temporary environmental situation is to be understood here as a situation in the everyday life of the user, which is characterized not only on the basis of acoustic features (as in the case of the hearing situation), but also by further features, for example, movement-related and/or location-related features.

In this case a number of acoustic indicators is initially ascertained on the basis of the first input signal, and possibly a further input signal of the hearing system. The further input signal can be generated here by a further input transducer of the hearing instrument, for example, in the case of a monaural hearing device configured for directional microphonics or also a binaural hearing aid as the hearing instrument. The acoustic indicator(s) are preferably ascertained in this case on the basis of a spectral analysis of the first input signal, and are configured in particular in such a way that they permit a conclusion about a so-called hearing situation, which exists at the moment of the recording of the first input signal.

In this case, a hearing situation directly, and/or at least one characteristic variable for a speech component and/or a separate speech activity of the user in the ambient sound recorded by the first input transducer, and/or a characteristic variable for a noise component, in particular a wind noise, in the ambient sound, and/or a characteristic variable for tonal signals in the ambient sound and/or a characteristic variable for a directionality (in particular its strength and precise direction) in the ambient sound are preferably ascertained as acoustic indicators. Furthermore, characteristic parameters with respect to the onset and/or the rhythm of a speech signal in the ambient sound, with respect to a steady state, and/or with respect to a spectral focal point of the ambient sound can also be used as acoustic indicators. A hearing situation may also be concluded on the basis of the mentioned characteristic parameters. In the case already discussed of a hearing instrument having a further input transducer which is configured for directional microphonics, a characteristic parameter for a directionality of the ambient sound can also be ascertained as an acoustic indicator.

A cardiovascular characteristic variable (in particular, a blood pressure and/or a pulse and/or a heart rate of the user), and/or a movement state of the user, and/or a position of the user and/or a body temperature of the user and/or a characteristic variable for a stress perception of the user are preferably ascertained here as peripheral indicators. A blood sugar level in particular can also be ascertained as a peripheral indicator. In the case of the heart rate or the pulse or the blood pressure, a photoplethysmography sensor (PPG sensor) is preferably used as the first sensor for ascertaining the peripheral indicator(s), in the case of the movement state, preferably an acceleration sensor, in the case of the position, preferably a GPS receiver and/or position information acquired via a WLAN connection, and in the case of the body temperature preferably a thermometer. Furthermore, a bioimpedance sensor, a sensor of electrodermal activity, an altimeter, and/or a brightness sensor can be used as the first sensor and a corresponding peripheral indicator can be generated on the basis of the respective sensor signal.

The peripheral indicators thus permit a finer assignment of the existing temporary environmental situations than would be possible by means of the acoustic indicator(s) alone. Thus, for example, a loud environment having many conversation components, a large amount of reverb, and many further interference noises, on the one hand, can be given by a conversation in a restaurant or the like (so-called “cocktail party” hearing situation), or also by the participation in a team sport in a hall. A delimitation may be performed significantly more effectively in this case on the basis of the peripheral indicators “movement state” and possibly blood pressure/pulse/heart rate than on the basis of acoustic indicators alone, for which the two situations are possibly more difficult to distinguish.

The first sensor can on the one hand be arranged in the hearing instrument here, or on the other hand in an auxiliary device, which is connectable to the hearing instrument and is associated with the hearing system, e.g., a smart phone, a smartwatch, or a fitness armband, or the like. In particular, the acoustic indicators can additionally also be ascertained on the basis of a further input signal, which is generated by a further electroacoustic input transducer of the hearing system (for example in the auxiliary device) from the ambient sound.

The temporary environmental situation existing at a specific point in time is thus characterized in particular in terms of the method by the acoustic and peripheral indicators ascertained for this point in time. In particular, similarities and/or differences from other temporary environmental situations and classifications into individual classes are in relation to said acoustic and peripheral indicators.

It is now ascertained on the basis of the respectively ascertained acoustic and peripheral indicator(s) whether the temporary environmental situation existing at a point in time falls in a class from a plurality of known classes for temporary environmental situations. Such a class is preferably characterized here in that it has intervals as value ranges for individual acoustic and peripheral indicators, if they can assume continuous values according to their nature (e.g., signal-to-noise ratio, percentage speech component, blood pressure, pulse/heart rate, etc.), so that a temporary environmental situation can only be in a class if at least the continuous ones of its acoustic and peripheral indicators are in the intervals correspondingly defined in each case for the class.

Alternatively thereto, or also for individual ones of the indicators, reference values and distance relations can be defined, i.e., for a class, individual reference values of specific indicators are defined in each case, and in addition in the parameter space defined by the respective indicators, distance relations are defined with respect to the reference values or also for vectors of the indicators with respect to a corresponding vector having the reference values. Comparable distance measures may be defined here for noncontinuous indicators.

A class for a temporary environmental situation is thus given by a typical situation in the everyday life of the user, so that the characterizing features of this situation, except for small deviations (represented, for example, by the above-mentioned intervals), cause an existence of said situation to be recognized (and thus in particular also a delimitation from other typical situations or classes). The existing daily routine environmental situation thus in particular falls into one of the known classes if the presently ascertained acoustic and peripheral indicators fall in the intervals of the associated indicators correspondingly defined for the known class, and/or other distance criteria meet values predefined with respect to the class.

A known class of temporary environmental situations is preferably assigned a plurality of parameter values for the corresponding signal processing parameters in this case, i.e., preferably in the case that the existing temporary environmental situation falls in one of the known classes, the signal processing of at least the first input signal (and possibly further input signals) to form the output signal is carried out according to the parameter values defined for the class.

If the existing temporary environmental situation does not fall into one of the known classes here, a new class is thus defined on the basis of the presently ascertained acoustic indicator(s) and on the basis of the presently ascertained peripheral indicator(s), so that the newly defined class is characterized by said indicators. Distance and/or similarity relations of the acoustic and peripheral indicators are preferably also defined here, so that a later existing temporary environmental situation also still falls into the newly defined class if a deviation from the corresponding value of the indicator used for the definition of the class exists for at least one of the indicators, but this deviation can be neglected with respect to the overall value range and/or to the relation to other classes.

In the definition of the new class, the plurality of signal processing parameters is now assigned a corresponding plurality of parameter values, according to which the first input signal (and possibly further ones) is to be processed to form the output signal if, at a later point in time, a temporary environmental situation exists which falls in the newly defined class on the basis of its acoustic and peripheral indicators (thus if said indicators have sufficient similarity to those values of the corresponding indicators which were used for the definition of the class).

The described procedure permits the user upon a recognition of a new temporary environmental situation by the hearing system, which does not fall in a known class, to define such a new class for later use. The user can thus define individual classes, individually tailored to his everyday life and his daily routine, and have corresponding parameter values assigned for the signal processing parameters, without having to visit a hearing aid acoustician or the like for this purpose.

Preferably, a first confirmation inquiry is directed to the user here for the definition of the new class by an operating device of the hearing system, for example via operating elements on the hearing instrument and/or on the above-mentioned auxiliary device (preferably via a touchscreen as in a smart phone, for example), and the definition is performed in case of a confirmation of the inquiry. If the existing temporary environmental situation is recognized as not compatible with any of the known classes according to its acoustic and peripheral indicators, the inquiry can be overlaid as to whether the user is presently located in a novel situation, and a new class is accordingly to be defined. If the user confirms this inquiry, the class is defined. If not, the definition does not take place.

Although the definition of the new class can also take place automatically and an interaction with the user is not absolutely required for this purpose, such an interaction in the form described here of the confirmation inquiry thus provides the advantage of dealing better with the individual requirements of the user. Thus, for example, not everything which was automatically recognized as an independent new class is also actually of importance for the user.

In the definition of the new class for the assignment of the plurality of parameter values, a similarity value of the new class with at least one existing class is advantageously ascertained, and the plurality of parameter values for the new class is assigned on the basis of the similarity value and on the basis of the plurality of parameter values of the at least one existing class. The similarity value can be formed, for example, on the basis of a distance relation of a vector of the values of the individual presently ascertained acoustic and peripheral indicators to a corresponding vector of indicators of said existing class or classes.

For example, in a vector space which is spanned by individual indicators, for the vector of the presently ascertained indicator values, the “closest” classes can be determined on the basis of corresponding vectors (the entries of which can each be given, for example, by reference values for the respective class) via a distance relation in the vector space, and said similarity value also on the basis of the distance relation (for example via a predetermined scale, which assigns a higher similarity value to a lower distance according to the distance relation). The plurality of parameter values can then each be assigned to the signal processing parameters, in that the corresponding parameter values of the closest class or classes, each weighted on the basis of the similarity measure, are used for the assignment.

In the definition of the new class, for the assignment of the plurality of parameter values, the ascertained acoustic and peripheral indicator(s) are expediently transmitted by means of a communication device of the hearing system to a computing system, separate from the hearing system, having a database (in particular to a central computing device having a central or decentral database, for example a cloud server), in which a plurality of class definitions is stored, wherein the plurality of parameter values for the new class are assigned by the computing system on the basis of the ascertained acoustic and peripheral indicator(s) and on the basis of the class definitions. The access to class definitions which were performed, for example, for other users (by these users or also by professional hearing aid acousticians), and the associated indicators, facilitates the new definition of classes. In particular, the assignment takes place here in the computing system by means of an artificial intelligence, for example an artificial neural network or the like, by which the new definition can be further simplified. The individual stored class definitions preferably also comprise the corresponding parameter values in this case.

Advantageously, in this case audiological and/or biometric data of the user are additionally transmitted to the computing system, wherein further audiological and/or biometric data associated with each class definition of a person assigned to the respective class definition are stored in the database, and wherein the plurality of parameter values for the new class are additionally assigned by the computing system on the basis of the audiological and/or biometric data of the user and on the basis of the audiological and/or biometric data of the persons assigned to the respective class definitions.

This means in particular that for the class definition, in addition to the respective parameter values which are assigned to the saved classes defined on the basis of the acoustic and peripheral indicators, in addition audiometric data (for example audiograms) and/or biometric data (e.g., with respect to age, sex, possibly occupation, chronic illnesses, etc.) of those persons for which each of the classes were defined, are also acquired and stored. The definition of the new class for the existing indicators can then additionally be carried out on the basis of a comparison of the audiological or biometric data, so that preferably those stored class definitions are taken into consideration more strongly for which the audiological or biometric data of the relevant person have a higher similarity to those of the user of the hearing instrument.

It has proven to be furthermore advantageous if upon the definition of the new class, for the assignment of the plurality of parameter values, in each case preliminary parameter values are assigned, and the first input signal is processed according to the preliminary parameter values for the plurality of signal processing parameters to form the output signal, wherein the output sound signal corresponding to the output signal thus generated is supplied to a sense of hearing of the user, and wherein a second confirmation inquiry is directed to the user, and in the case of a corresponding confirmation, the preliminary parameters are defined as the plurality of parameter values for the definition of the class. This comprises in particular that a class definition is presented to the user for the temporary environmental situation having the associated parameter values in a type of test operation, and this user can decide via the second confirmation inquiry whether he wishes to retain these preliminary parameter values, which are used in the test operation.

In case of a rejection of the second confirmation inquiry, an inquiry with respect to a change of a parameter value for a certain one of the signal processing parameters is preferably directed to the user here, wherein a change on said parameter value for the specific signal processing parameter is performed on the basis of a corresponding input of the user. This means in particular that in case of a rejection of the second confirmation inquiry—thus if the user negatively assesses the described test operation of the new class definition having the corresponding preliminary parameters—the user receives the option of deliberately adapting individual signal processing parameters himself. The possibility can be opened up in this case, for example, of selecting corresponding signal processing parameters for an adaptation, and then setting them either by proposals for parameter values to be confirmed or directly via a type of regulator function of a user interface (for example an app), by means of which the respective confirmation inquiry is output to the user. After completion of the setting of the parameter values for the desired signal processing parameters, the new definition of the class can then be stored, for example.

With the above and other objects in view there is also provided, in accordance with the invention, a novel hearing system, which comprising a hearing instrument with at least one input transducer, a signal processing unit, an output transducer, and a first sensor. The input transducer is thereby configured to generate an input signal from an ambient sound, wherein the signal processing unit is configured to process the input signal according to a plurality of signal processing parameters to form an output signal, wherein the output transducer is configured to convert the output signal into an output sound signal, wherein the first sensor is configured to ascertain a number of peripheral indicators for an existing temporary environmental situation of a user of the hearing instrument, and wherein the hearing instrument is configured to adapt the plurality of signal processing parameters according to the above-described method.

The hearing system according to the invention shares the advantages of the method according to the invention. The advantages indicated for the method and for its refinements can be transferred accordingly to the hearing system. Other, or additional, features which are considered as characteristic for the invention are set forth in the appended claims.

Although the invention is illustrated and described herein as embodied in a method for a hearing system for adapting a plurality of signal processing parameters of a hearing instrument of the hearing system, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 shows a block diagram of a hearing system having a hearing aid and a smart phone, which is configured to acquire a general situation in the daily routine of a user; and

FIG. 2 shows a block diagram of the sequence of a method to define signal processing parameters for the hearing aid depending on the acquired situation by means of the hearing system shown in FIG. 1.

Parts and variables corresponding to one another are provided with the same reference signs and symbols throughout the figures.

### DETAILED DESCRIPTION OF THE INVENTION

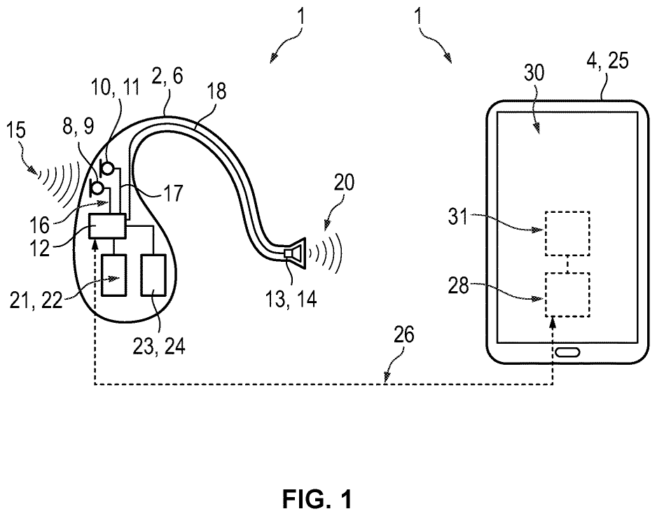

Referring now to the figures of the drawing in detail and first, in particular, to FIG. 1 thereof, there is shown a schematic diagram of a hearing system 1, which includes a hearing instrument 2 and an auxiliary device 4. The hearing instrument 2 is designed in the present case as a hearing aid 6, and accordingly has a first electroacoustic input transducer 8, a second electroacoustic input transducer 9, a signal processing unit 12 connected to both said input transducers 8, 10, and an electroacoustic output transducer 14 connected to the signal processing unit 12. The first and the second electroacoustic input transducer are provided here by a first microphone 9 and a second microphone 11, respectively, and the electroacoustic output transducer 14 is provided by a loudspeaker 13. The first and second microphones 9, 11 are configured to generate a first input signal 16 or a second input signal 17, respectively, from an ambient sound 15. The first input signal 16 and the second input signal 17 are processed in the signal processing unit 12 to form an output signal 18, which is converted by the loudspeaker 13 into an output sound signal 20. In the processing of the two input signals 16, 17 in the signal processing unit 12, in particular a hearing loss of a user (not shown in greater detail) of the hearing aid 6 can be taken into consideration in that the hearing loss is at least partially compensated for by a frequency band-dependent amplification and/or compression of the input signals 16, 17. For this purpose, individual signal processing parameters for the processing of the first and the second input signal 16, 17 are set to corresponding parameter values in the signal processing unit 12 in a dependence still to be described on external conditions

Furthermore, the hearing aid 6 has, as a first sensor 21, a PPG sensor 22, which is also connected to the signal processing unit 12. The PPG sensor 22 is configured in particular to measure a heart rate and/or a blood pressure of the user. The hearing aid 6 additionally has in the present exemplary embodiment, as a further sensor 23, an acceleration sensor 24, which is also connected to the signal processing unit 12, and by means of which a movement change of the user of the hearing aid 6 can be acquired. A movement of the user can thus also be acquired by an integration of the acceleration in the signal processing unit 12.

In a way still to be described, said signal processing parameters for the processing of the two input signals 16, 17 are set in dependence on external conditions to corresponding parameter values. In particular, it is to be made possible for the user here to define such dependencies on the external conditions, thus a relationship between the conditions and the corresponding assignment of parameter values for the signal processing parameters, himself according to his individual requirements. In particular the auxiliary device 4, designed in the present case as a smart phone 25, is used for this purpose, which is connectable via a Bluetooth connection 26 to the hearing aid 6. For this purpose, the hearing aid 6 and the smart phone 25 each have correspondingly configured transceiver devices (not shown in greater detail in FIG. 1) (for example antennas) for establishing said Bluetooth connection 26. The smart phone 25 additionally has a processor unit 28 for processing steps described hereinafter, and a touchscreen 30, by means of which user inputs can be performed by the wearer of the hearing aid 6 or the system 1 via a corresponding application. In addition, the smart phone 25 has a GPS sensor 31, which is connected to the processor unit 28 and is to be used as a further sensor for the acquisition of the external conditions.

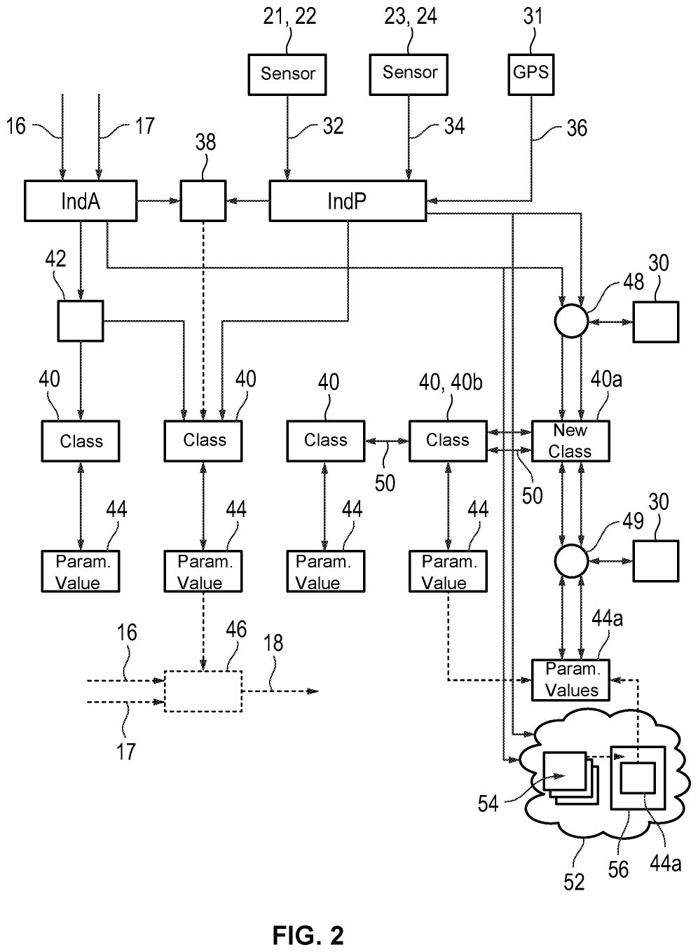

FIG. 2 schematically shows in a block diagram the sequence of a method, by means of which the signal processing parameters of the hearing aid 6 according to FIG. 1 are assigned individual parameter values in dependence on external conditions. On the basis of the first input signal 16 and the second input signal 17, initially acoustic indicators IndA are obtained by an analysis of said signals in the signal processing unit 12, which permit conclusions about the external conditions such as hearing situation or also environment in general. For this purpose, the two input signals 16, 17 are preferably decomposed into individual frequency bands. The acoustic indicators can preferably directly comprise a hearing situation here, or also a characteristic variable for a speech component, for an ego speech activity of the user, for a noise component, or for tonal signals in the ambient sound. Furthermore, the directionality of sound signals of individual sound sources in the ambient sound is preferably also used by means of directional microphonics (by corresponding processing of the two input signals 16, 17), which is in particular advantageous for a recognition of the existing hearing situation.

Furthermore, peripheral indicators IndP are then also acquired by means of the first sensor 21 and the further sensor 23. The first sensor 21, designed as a PPG sensor 22, acquires in this case as peripheral indicators IndP at least one cardiovascular characteristic variable 32 of the user, thus, for example, a blood pressure and/or a pulse rate. The further sensor 23, designed as an acceleration sensor 24, acquires as a further peripheral indicator IndP movement changes of the user, due to which in particular conclusions are possible about physical activity 34, for example sporting activity or also physical work. The corresponding peripheral indicator IndP can also be given here by a more detailed evaluation of the acquired movement changes with respect to the precise type of the physical activity (see above). In addition, a position 36 of the user can be acquired by the GPS sensor 31 of the smart phone 25 as a further peripheral indicator IndP.

The acoustic indicators IndA and the peripheral indicators IndP define a temporary environmental situation 38 here. This temporary environmental situation 38 in particular represents a characterization of the external conditions according to the entirety of its acquired features. It is not checked on the basis of the acoustic indicators IndA and the peripheral indicators IndP whether the existing temporary environmental situation 38 falls in a previously defined class 40 for temporary environmental situations. Such classes 40 for temporary environmental situations can be understood here in particular as an expansion of the concept of the hearing situation to nonacoustic features (acquired in the present case on the basis of the peripheral indicators IndP) for a further differentiation.

The check as to whether the existing temporary environmental situation 38 falls into one of said classes 40 can also take place in a cascaded manner, in that, for example, solely a hearing situation 42 is initially ascertained on the basis of the acoustic indicators IndA, which can be given, however, by several of the classes 40 for temporary environmental situations, and it is subsequently checked on the basis of the peripheral indicators IndP whether the existing temporary environmental situation 38 can be assigned to one of the relevant classes 40 for the existing hearing situation 42.

Each individual one of the classes 40 for temporary environmental situations is characterized here, on the one hand, on the basis of the relevant acoustic and peripheral indicators IndA, IndP, wherein preferably a distance and/or similarity measure is also defined, so that for acoustic and peripheral indicators IndA, IndP existing at a given point in time, it can be ascertained on the basis of the distance or similarity measure with respect to the corresponding indicators of the predefined classes 40 in which class 40 the corresponding temporary environmental situation 38 existing at the point in time falls. Such a distance or similarity measure can also be defined for continuous indicators, for example, on the basis of corresponding intervals for the values of the respective indicators; a distance measure in a high-dimensional vector space is also conceivable in this case (a class is then preferably defined via a base vector having individual values of the respective indicators as vector entries).

On the other hand, each class 40 also comprises an assignment of a plurality of parameter values 44 to the corresponding signal processing parameters 46 of the signal processing unit 12. This means: The signal processing parameters 46, according to which the two input signals 16, 17 are processed to form the output signal 20 in the signal processing unit 12, are set as a function of the presently existing class 40 for temporary environmental situations (identified on the basis of the acoustic and peripheral indicators IndA, IndP) to respective parameter values 44, which were defined or established beforehand for the present class 40.

If it is now however established on the basis of the acoustic indicators IndA and the peripheral indicators IndP (for example via a distance or similarity measure, see above) that the currently existing temporary environmental situation 38 does not fall into any of the known classes 40, a new such class 40a is thus defined. For the definition, the currently existing values of the acoustic and peripheral indicators IndA, IndP are used here. On the one hand, the definition can take place automatically. On the other hand, a first confirmation inquiry 48 can also be output to the user via the touch screen 30 of the smart phone 25 as to whether this user wishes a definition of a new class 40a for temporary environmental situations, and the definition can be performed upon the confirmation of the inquiry (for example, the user can reject the first confirmation inquiry 48 if the current situation does not represent a repeating temporary environmental situation with a high level of certainty).

If a new class 40a for temporary environmental situations is now defined, an assignment of the corresponding parameter values 44a for the associated signal processing parameters 46 also takes place. This assignment can take place, on the one hand, on the basis of an acoustic analysis of the surroundings (thus by means of the input signals 16, 17) or on the basis of an ascertained hearing situation 42, wherein if necessary the user can perform fine tuning via the touch screen 30.

In particular, a test operation of the signal processing using preliminary parameter values can be carried out for this purpose, and a second confirmation inquiry 49 can be output to the user. Upon a confirmation, the preliminary parameter values are established for the definition of the new class 40a. Upon a rejection, the user can adapt individual signal processing parameters 46, for example by successive inquiries by the hearing system.

However, the parameter values 44a for the new class 40a can also be obtained in that, on the basis of the acoustic and peripheral indicators IndA, IndP for the currently existing temporary environmental situation 38, a similarity value 50 to other classes 40 for temporary environmental situations is formed (preferably in each case with respect to a reference vector of the relevant class having corresponding indicator values). On the basis of the similarity value 50, a most similar class 40b (or also a second-most similar class, etc.) to the existing temporary environmental situation 38 can then be determined. The parameter values 44a of the new class 40a can then be obtained on the basis of the parameter values 44 of the most similar class 40b. The above-mentioned test operation having preliminary parameter values and the second confirmation inquiry 49 can also be carried out here.

In particular, the similarity value 50 can also already be used here for the recognition of the new class 40a. For example, this can take place in such a way that for the existing temporary environmental situation 38, the similarity value 50 is ascertained with respect to all known classes 40. If the greatest similarity value 50 is above a predetermined limiting value (with suitable norming, this limiting value is to be selected close to 1), by definition the existing temporary environmental situation 38 falls into the known class 40. However, if the similarity value 50 having the existing temporary environmental situation 38 does not reach the limiting value for any of the known classes 40, the new class 40a is thus defined accordingly, wherein for the parameter values 44a, those of the most similar class 40b (the class having the greatest similarity value 50) can be used and can be suitably modified by the user if necessary.

The assignment of the parameter values 44a of the new class 40a can also take place in that the acoustic and peripheral indicators IndA, IndP are transferred to a computing system 52 having a database (not shown in greater detail), which can be provided, for example, by a cloud server. A large number of class definitions 54 for temporary environmental situations, as were already carried out for other persons (in particular by this person himself) are stored in said database. In particular, the respective indicators IndA, IndP and the associated parameter values 44 are thus stored here for the individual class definitions 54, thus for the definitions of classes 40 for temporary environmental situations performed for or by other persons. On the basis of the stored class definitions 54, a suitable set of parameter values 44a for the new class 40a can be ascertained by a computing unit 56 connected to the database of the computing system 52, which as part of the computing system 52 can also be implemented on the physical infrastructure of the cloud server, and transferred back to the hearing system 1. The selection of these suitable parameter values 44a can in particular also take place here in a similar manner as described above on the basis of a similarity measure, wherein the similarity of the existing temporary environmental situation is now checked with respect to the stored class definitions.

In particular, however, for the individual class definitions 54, audiological data (for example an audiogram) and/or biometric data (e.g., age, sex, relevant illnesses, etc.) of the respective persons can also be stored, for whom the class definitions 54 were performed. Corresponding audiological and/or biometric data of the user of the hearing instrument 2 are then also to be transferred to the database of the computing system 52 for the ascertainment of the parameter values 44a of the new class 40a; these data can preferably be stored in the smart phone 25, by means of which the transfer of said data and in principle also the acoustic and peripheral indicators can take place.

The above-mentioned test operation using preliminary parameter values and the second confirmation inquiry 49 can also be initially carried out here for the final determination of the parameter values 44a for the definition of the new class 40a.

Although the invention was illustrated and described in more detail by the preferred exemplary embodiment, the invention is not thus restricted by the disclosed examples and other variations can be derived therefrom by a person skilled in the art without leaving the scope of protection of the invention.

The following is a summary list of reference numerals and the corresponding structure used in the above description of the invention:
1 hearing system
2 hearing instrument, hearing device
4 auxiliary device
6 hearing aid
8 first (electroacoustic) input transducer
9 first microphone
10 second (electroacoustic) input transducer
11 second microphone
12 signal processing unit
13 loudspeaker
14 (electroacoustic) output transducer
15 ambient sound
16 first input signal
17 second input signal
18 output signal
20 output sound signal
21 first sensor
22 PPG sensor
23 further sensor
24 acceleration sensor
25 smart phone
26 Bluetooth connection
28 processor unit
30 touchscreen
31 GPS sensor
32 cardiovascular characteristic variable
34 physical activity
36 position
38 temporary environmental situation
40 class for temporary environmental situations
40a new class (for temporary environmental situations)
42 hearing situation
44 parameter values
44a parameter values (of the new class)
46 signal processing parameters
48 first confirmation inquiry
49 second confirmation inquiry
50 similarity value
52 computing system
54 class definition
56 computing unit
IndA acoustic indicators
IndP peripheral indicators

1. A method for adapting a plurality of signal processing parameters of a hearing instrument of a hearing system, the method comprising:
processing a first input signal, which is generated by a first input transducer of the hearing instrument from an ambient sound, to form an output signal, and converting the output signal by an output transducer of the hearing instrument into an output sound signal;
determining, based on the first input signal, a number of acoustic indicators for an existing temporary environmental situation of a user of the hearing instrument;
determining, based on at least one first sensor of the hearing system, a number of peripheral indicators for the existing temporary environmental situation;
on a basis of one or more of the acoustic indicators and on a basis of one or more of the peripheral indicators, ascertaining whether the existing temporary environmental situation falls in a class from a plurality of known classes for temporary environmental situations; and
when the existing temporary environmental situation does not fall into any of the known classes for temporary environmental situations:
defining a new class for temporary environmental situations, the new class being characterized by the respectively ascertained acoustic indicators and peripheral indicators; and
assigning to the plurality of signal processing parameters a corresponding plurality of parameter values, according to which the first input signal is to be processed to form the output signal upon a later existence of a temporary environmental situation from the new class.

2. The method according to claim 1, which comprises ascertaining at least one acoustic indicator selected from the group consisting of:
a hearing situation;
a characteristic variable for a speech component and/or an own speech activity of the user in the ambient sound;
a characteristic variable for a noise component in the ambient sound;
a characteristic variable for tonal signals in the ambient sound; and
a characteristic variable for a directionality in the ambient sound.

3. The method according to claim 1, which comprises ascertaining at least one peripheral indicator selected from the group consisting of:
a cardiovascular characteristic variable, and/or
a movement state of the user;
a position of the user;
a body temperature of the user; and
a characteristic variable for a stress perception of the user.

4. The method according to claim 3, wherein the first sensor for ascertaining the at least one peripheral indicator is a sensor selected from the group consisting of:
a photoplethysmography sensor;
an acceleration sensor;
a GPS receiver;
a thermometer;
a bioimpedance sensor;
a sensor of electrodermal activity; and
an altimeter.

5. The method according to claim 1, which comprises, before a new class is defined, directing a confirmation inquiry to the user by an operating device of the hearing system, and defining the new class when the inquiry is confirmed by the user.

6. The method according to claim 1, wherein the step of defining the new class comprises ascertaining a similarity value of the new class with at least one existing class, and assigning the plurality of parameter values for the new class on a basis of the similarity value and on a basis of the plurality of parameter values of the at least one existing class.

7. The method according to claim 1, wherein the step of defining the new class comprises:
transferring the ascertained acoustic and peripheral indicators by way of a communication device of the hearing system to a computing system that is separate from the hearing system and that has a database in which a plurality of class definitions are stored; and
assigning the plurality of parameter values for the new class by the computing system on the basis of the ascertained acoustic and peripheral indicators and on the basis of the class definitions.

8. The method according to claim 7, which comprises:
transmitting additional audiological and/or biometric data of the user to the computing system;
wherein further audiological and/or biometric data, associated with each class definition, of a person assigned to the respective class definition are stored in the database; and
additionally assigning the plurality of parameter values for the new class by the computing system on a basis of the audiological and/or biometric data of the user and on a basis of the audiological and/or biometric data of the persons assigned to the respective class definitions.

9. The method according to claim 1, wherein the step of defining the new class comprises:
assigning respective preliminary parameter values and processing the first input signal with the preliminary parameter values for the plurality of signal processing parameters to form the output signal;
supplying the output sound signal corresponding to the output signal thus generated to a sense of hearing of the user; and
directing a confirmation inquiry to the user and, upon receiving a confirmation, establishing and assigning the preliminary parameters as the plurality of parameter values for the definition of the new class.

10. The method according to claim 9, which comprises:
upon receiving a rejection of the confirmation inquiry, directing an inquiry with respect to a change of a parameter value for a specific one of the signal processing parameters to the user; and
changing said parameter value for the specific signal processing parameter based on a corresponding input of the user.

11. A hearing system, comprising:
a hearing instrument having at least one input transducer, a signal processing unit, and an output transducer;
said at least one input transducer being configured to generate an input signal from an ambient sound;
said signal processing unit being configured to process the input signal according to a plurality of signal processing parameters to form an output signal;
said output transducer being configured to convert the output signal into an output sound signal; and
a first sensor configured to ascertain a number of peripheral indicators for an existing temporary environmental situation of a user of the hearing instrument; and
wherein the hearing system is configured to adapt the plurality of signal processing parameters by performing the method according to claim 1.
